(19) United States
(12) Patent Application Publication
(10) Pub. No.: US 2025/0137055 A1
(43) Pub. Date: May 1, 2025

(54) Method of Predicting a Patient's Benefit From Therapy with an Immune Checkpoint Inhibitor

(71) Applicant: Eberhard-Karls-Universität Tübingen Universitätsklinikum, Tuebingen (DE)

(72) Inventors: Oltin-Tiberiu Pop, St. Gallen (DE); Teresa Amaral, Tübingen (DE); Lukas Flatz, Muri Bei Bern (CH)

(21) Appl. No.: 18/384,708

(22) Filed: Oct. 27, 2023

(51) Int. Cl. C12Q 1/6886; C12Q 1/6869; G01N 33/574

(52) U.S. Cl. CPC C12Q 1/6886; C12Q 1/6869; G01N 33/5743; G01N 33/57492; C12Q 2600/106; C12Q 2600/158; G01N 2333/485

(57) ABSTRACT

The present invention relates to a method of predicting a patient's benefit from therapy with an immune checkpoint inhibitor (ICI), a method of treatment of a cancer patient with an ICI, and to a method of predicting the likelihood of a cancer patient's relapse after treatment with an ICI.

The present invention relates to a method of predicting a patient's benefit from therapy with an immune checkpoint inhibitor (ICI), a method of treatment of a cancer patient with an ICI, and to a method of predicting the likelihood of a cancer patient's relapse after treatment with an ICI.

### FIELD OF THE INVENTION

The present invention relates to the field of molecular biology and molecular medicine, more particular to the field of molecular diagnostics and prognostics and therapy.

### BACKGROUND OF THE INVENTION

Melanoma is the most dangerous type of skin cancer. Globally, in 2012, it newly occurred in 232,000 people. In 2015, 3.1 million people had active disease, which resulted in 59,800 deaths. Australia and New Zealand have the highest rates of melanoma in the world. High rates also occur in Northern Europe and North America, while it is less common in Asia, Africa, and Latin America. In the United States, melanoma occurs about 1.6 times more often in men than women. Melanoma has become more common since the 1960s in areas mostly populated by people of European descent.

The primary cause of cutaneous melanoma is ultraviolet (UV) light exposure in individuals with low levels of the skin pigment melanin. The UV light may be from the sun or other sources, such as tanning devices. Those with many moles, a family history of melanoma, and immunosuppression are at greater risk. A number of rare genetic conditions, such as xeroderma pigmentosum, also increase the risk. Diagnosis is made by biopsy and histopathological examination of any skin lesion suspected of being potentially cancerous.

Using sunscreen and avoiding UV light may prevent melanoma. Treatment typically consists of surgical removal of the melanoma and potentially affected surrounding tissue. In those with thicker lesions, nearby lymph nodes (sentinel lymph nodes) may be tested for the presence of tumor cells (metastasis). Most people are cured if spread has not occurred. For those in whom melanoma has spread, immunotherapy or targeted therapy may improve survival. With treatment, the five-year survival rates in the United States are 99% among those with localized disease, 65% when the disease has spread to lymph nodes, and 25% among those with distant spread. The likelihood that melanoma will recur or spread depends on primary tumor thickness, whether or not the overlying skin has broken down (ulceration), and the presence of local spreading (sentinel lymph node involvement).

The development of immune checkpoint inhibitors (ICI) has transformed the treatment of melanoma. Blockade of inhibitory receptors, CTLA-4 and PD-1, enhances T-cell-mediated antitumor immune responses, leading to improved survival and durable responses in patients. Based on their mechanism of action, immune checkpoint inhibitors can also induce immune-related adverse events that require careful monitoring and prompt treatment.

Adjuvant therapy with ICI became standard for resected stage III/IV melanoma after the results from the Checkmate 238 and Keynote 054 studies. However, 30-40% of patients relapse despite adjuvant therapy; Weber et al. Adjuvant Nivolumab versus Ipilimumab in Resected Stage III or IV Melanoma. New England Journal of Medicine. 2017; 377 (19): 1824-1835; Eggermont et al. Adjuvant Pembrolizumab versus Placebo in Resected Stage III Melanoma. New England Journal of Medicine. 2018; 378 (19): 1789-1801.

Therefore, there is a need to identify biomarkers that are predictive of response.

Considering this background, an object underlying the invention is to provide a method for predicting a patient's benefit from therapy with an ICI, which involves the use of an appropriate biomarker. Another object of the invention is to provide a method of treating a patient in need, in particular a cancer patient, in which the decision to administer an ICI is made dependent on potential responsiveness of the patient to ICI.

By means of said methods the disadvantages of the state of the art are avoided or at least significantly reduced, which are that currently ICIs are largely routinely administered for certain forms of cancer, such as malignant melanoma in resected stage Ill or IV, without knowing whether or not such therapy is even useful.

### SUMMARY OF THE INVENTION

The present invention provides a method of predicting a patient's benefit from therapy with an immune checkpoint inhibitor (ICI), comprising the following steps:
1) Providing a biological sample from the patient;
2) Determining the expression level of epidermal growth factor receptor (EGFR) on the biological material to obtain C_{EGFR}^{Pat.};
3) Comparing C_{EGFR}^{Pat.} with the expression level of EGFR in a reference biological sample C_{EGFR}^{Ref.},
4) Predicting
   no therapeutic benefit if C_{EGFR}^{Pat.}>C_{EGFR}^{Ref.}.
   a therapeutic benefit if C_{EGFR}^{Pat.}≤C_{EGFR}^{Ref.}.

The present invention also provides a method of treatment of a cancer patient with an immune checkpoint inhibitor (ICI), comprising the following steps:
1) Providing a biological sample from the patient;
2) Determining the expression level of epidermal growth factor receptor (EGFR) on the biological material to obtain C_{EGFR}^{Pat.};
3) Comparing C_{EGFR}^{Pat.} with the expression level of EGFR in a reference biological sample C_{EGFR}^{Ref.}
   4) Refraining from administering the ICI to the patient if C_{EGFR}^{Pat.}>C_{EGFR}^{Ref.};
   or
   Administering the ICI to the patient if C_{EGFR}^{Pat.}≤C_{EGFR}^{Ref.}.

The present invention further provides a method of predicting the likelihood of a cancer patient's relapse after treatment with an immune checkpoint inhibitor (ICI), comprising the following steps:
1) Providing a biological sample from the patient;
2) Determining the expression level of epidermal growth factor receptor (EGFR) on the biological material to obtain C_{EGFR}^{Pat.};
3) Comparing C_{EGFR}^{Pat.} with the expression level of EGFR in a reference biological sample C_{EGFR}^{Ref.}
4) Predicting a high likelihood for the patient to relapse if C_{EGFR}^{Pat.}>C_{EGFR}^{Ref.};
   or
   Predicting a low likelihood for the patient to relapse if C_{EGFR}^{Pat.}≤C_{EGFR}^{Ref.}.

The features, characteristics, advantages and embodiments set forth in the following apply equally to all methods and all objects of the invention.

According to the invention, an “immune checkpoint inhibitor” (ICI) refers to a molecule that inhibits an immune checkpoint, i.e., key regulators of the immune system that when stimulated can dampen the immune response to an immunologic stimulus. ICIs can block inhibitory checkpoints, restoring immune system function. Currently approved checkpoint inhibitors target the molecules CTLA4, PD-1, and PD-L1. Typical immune checkpoint inhibitors are e.g., antibodies against CTLA-4 (e.g., ipilimumab), PD-1 (e.g. nivolumab, pembrolizumab, cemiplimab) and PD-L1 (e.g. atezolizumab, durvalumab and avelumab). After the ICI has been administered, the ICI binds to these proteins, which act as immune checkpoints. As a result, the cells that carry one of these proteins on the cell surface and bind the ICI are temporarily (or as long as the therapeutic ICI is circulating in the body) attacked by immune cells and removed from the body by macrophages (temporary cell depletion). These processes lead to an increased anti-tumor immune response, so that tumor immune evasion is counteracted.

According to the invention, a “biological sample” refers to material originating from the patient under investigation, such as a human or animal subject, that can potentially comprise receptors either as expression product, i.e. protein, or as coding material, e.g. DNA, RNA, in particular mRNA, such as a biological cell, cell tissue, organs, etc.

According to the invention, “epidermal growth factor receptor” (EGFR; ErbB-1; HER1 in humans) is a transmembrane protein that is a receptor for members of the epidermal growth factor family (EGF family) of extracellular protein ligands. The epidermal growth factor receptor is a member of the ErbB family of receptors, a subfamily of closely related receptor tyrosine kinases. According to the invention, both wild-type and mutant EGFR are encompassed.

According to the invention the expression level is a measure of the strength of the presence of the EGFR in the biological sample under examination, either in the form of the receptor protein and/or the coding material, i.e., the encoding DNA and/or RNA or mRNA. The expression level can be increased if the amount of EGFR and/or coding material is increased. However, it can also be increased if the amount of EGFR is increased but the coding material is not, but the latter is subject to increased transcription and/or translation. The biological causes of increased expression of EGFR are insignificant according to the invention. They can be based on a mutation in the coding sequence of EGFR. However, they can also be based on a mutation of members of the transcriptional control, such as promoters, enhancers, silencers, etc. They can also result from delayed protein degradation of EGFR. Other causes are also conceivable.

According to the invention, C_{EGFR}^{Pat.} denotes the expression value or level of EGFR in the biological sample as determined in step 2. In one embodiment of the invention, it can be the concentration and/or amount of EGFR or the percentage of tumor cells classified as positive for EGFR.

The quantitative assessment of EGFR expression in the biological material, e.g., in melanoma tumors, can be performed on whole slide scans of EGFR-stained biological material, e.g., melanoma tumors by using appropriate software, such as the QuPath v0.2.3 software platform for whole slide image analysis as follows: Whole slide scans are individually loaded into the software, image stain vectors and background estimates are applied in a representative area containing background along with examples of strong nuclear counterstaining and chromogen staining, “Positive Cell Detection” command is used to identify cells across the selected region of interest (ROI)—defined as the whole tumor tissue within the stained tissue section—based upon nuclear staining, a random trees classifier (RTrees) is interactively trained to distinguish tumor cells from all other detections, cells are classified as positive or negative based upon a single intensity threshold applied to the maximum optical density of the detection chromogen within the cell.

According to the invention, a reference sample is understood to be a biological material that assuredly shows an average or normal expression level of EGFR.

C_{EGFR}^{Ref.} is the level or threshold of EGFR expression that must be exceeded in order for it to be predicted according to the invention that no therapeutic benefit can be expected due to administration of an ICI. The reference sample can, in one embodiment, originate from a healthy reference subject, then preferably from a corresponding tissue part. That is, if the patient's biological sample originates, for example, from a lymph node, the reference biological sample preferably also originates from a lymph node of the reference subject. In another embodiment, the reference sample originates from the patient itself, then preferably from a tissue part that shows a normal expression level of EGFR. It can also be a non-diseased tissue equivalent in this embodiment. For example, if the biological sample to be tested is from a lymph node affected by metastases, the reference sample is from a lymph node not affected by metastases. In an alternative embodiment, the reference sample can also be the patient's diseased tissue. In this embodiment, a negative prognosis, i.e., prediction of no therapeutic benefit, occurs if EGFR expression is detected in a certain proportion of the cells of the patient's biological material, i.e., a threshold value in this respect is exceeded. For example, if approximately 5% of the cells of the patient's biological material express EGFR, the prognosis is negative; if less than 5% of the cells of the patient's biological material express EGFR, the prognosis is positive. These principles apply to the selection of the reference tissue and is understandable to a skilled person.

In an embodiment, the expression level EGFR in a reference sample (C_{EGFR}^{Ref.}) is defined as the cut-off for EGFR expression that would best identify two groups of patients with higher relapse-free survival (RFS) difference; this cut-off is expressed as percentage of EGFR-positive tumor cells within the total number of tumor cells and is calculated using ROC curve analysis and Youden's index for EGFR positivity values assessed using quantitative digital pathology on two independent cohorts of patients diagnosed with melanoma

According to the invention, a “high likelihood” to relapse means more than 20%, preferably more than 30%, further preferably more than 40%, further preferably more than 50%, further preferably more than 60% likelihood that the patient re-develops cancer (e.g., the tumor, melanoma, metastases) after the ICI treatment. Accordingly, a “low likelihood” means that equal or less than 20%, preferably less than 15%, further preferably less than 10%, further preferably less than 5% likelihood that the patient re-develop the disease after the ICI treatment.

Using two independent cohorts of melanoma patients treated with immune checkpoint inhibitors, the inventors were able to determine that such patients who exhibited elevated EGFR expression in the metastases were more likely to relapse and have a significantly shorter lifespan compared to those patients who exhibited low EGFR expression in the metastases.

The inventors have recognized that the expression level of EGFR is a particularly suitable biological marker for a patient's responsiveness to therapy with an immune checkpoint inhibitor (ICI). This finding is particularly valuable. Thus, while ICIs are fundamentally promising therapeutics in the treatment of cancer, in particular melanoma, these agents are expensive and not all patients respond to therapy with ICIs (response rate up to 40%). Thus, due to the invention, patients who do not show responsiveness to ICI therapy can be spared the side effects of the treatment as well as the associated costs. This also relieves the burden on the healthcare system and counteracts any shortage of ICIs.

In an embodiment of the methods according to the invention, the biological sample comprises skin cancer tissue, including primary melanoma tissue, and preferably metastatic melanoma tissue.

With this measure, such a biological material is provided, which is particularly suitable for reliably determining the patient's responsiveness to ICI therapy. In particular, the inventors have found that EGFR expression is particularly high in relapse patients compared to long-term survivors, especially in therapy-naïve metastatic tissues, whereas in other tissues such differences are not as prominent. This measure therefore advantageously serves to improve the prognostic accuracy of patient responsiveness to ICI therapy.

In still another embodiment of the invention in step (4) no therapeutic benefit is predicted if at least approx. 5% of the cells of the biological sample from the patient express EGFR.

With this measure, an expression threshold is established to ensure that the patient's responsiveness to ICI therapy is reliably and accurately determined. This will advantageously prevent a patient from being misclassified as being susceptible to ICI therapy when in fact he/she is not.

According to the invention, “at least approx. 5%” means approx. 5%, approx. 6%, approx. 7%, approx. 8%, approx. 9%, approx. 10%, approx. 15%, approx. 20%, approx. 25%, approx. 35%, approx. 40%, approx. 45%, approx. 50%, approx. 55%, approx. 60%, approx. 65%, approx. 70%, approx. 75%, approx. 80%, approx. 85%, approx. 90%, approx. 95%, approx. 100%.

According to another embodiment of the invention the ICI is an anti-PD-1 antibody, including pembrolizumab, nivolumab, and cemiplimab.

In still another embodiment of the invention the ICI is an anti-CTLA4 antibody, including ipilimumab and tremelimumab.

In yet another embodiment the ICI is an anti-PD-L1 antibody, including atezolizumab, avelumab, and durvalumab.

By these measures according to the invention is adapted to the ICIs which are currently available. This embodiment, therefore, implements the method into the common ICI therapies.

In another embodiment of the invention the expression level is determined by immunohistochemistry (IHC).

Due to this measure, an established detection method is used which is well suited to determine EGFR expression levels at the protein or receptor level and is suitable for accurate and reliable determination of ICI responsiveness. This embodiment uses commercially available and high affinity anti-EGFR antibodies which are already validated and approved for routine histopathological diagnosis in pathology departments.

In yet another embodiment of the invention, the patient is suffering from melanoma, especially from stage III/IV.

Due to this measure, an adaptation of the methods according to the invention takes place precisely for that group of patients who can particularly benefit from ICI treatment, provided that they are responsive to ICI therapy. Therefore, due to this embodiment, a valuable tool is provided for one of the most important potential ICI treatment groups.

Another subject-matter of the invention is a method of treatment of a patient with metastatic melanoma, comprising the steps of:
measuring the expression level of epidermal growth factor receptor (EGFR) in metastatic melanoma cells from the patient to obtain C_{EGFR}^{Pat.};
comparing C_{EGFR}^{Pat.} with the expression level of EGFR in a reference biological sample C_{EGFR}^{Ref.}
predicting that the patient will benefit from treatment with an immune checkpoint inhibitor (ICI) if C_{EGFR}^{Pat.} Is less than or equal to C_{EGFR}^{Ref.};
subjecting the patient to a cancer treatment regimen that comprises administering the ICI to the patient.

Still another subject-matter of the invention relates to a method of treatment of patients with metastatic melanoma, the method comprising:
A. for each patient,
   i. measuring the expression level of epidermal growth factor receptor (EGFR) in metastatic melanoma cells from the patient to obtain C_{EGFR}^{Pat}; and
   ii. comparing C_{EGFR}^{Pat} with the expression level of EGFR in a reference biological sample C_{EGFR}^{Ref}:
B. treating the patients for melanoma, wherein
   i. for at least one of the patients, predicting that the at least one patient will benefit from treatment with an immune checkpoint inhibitor (ICI) if C_{EGFR}^{Pat} of said at least one patient is less than C_{EGFR}^{Ref.}; and treating said at least one patient with a cancer treatment regimen that comprises administering the ICI to the patient; and
   ii. for at least one other of the patients, predicting that the at least one other of the patients will not benefit from treatment with an immune checkpoint inhibitor (ICI) if C_{EGFR}^{Pat.} of said at least one other patient is equal or greater than C_{EGFR}^{Ref}; and treating said at least one other patient with an alternate cancer treatment regimen that does not include the ICI.

In an embodiment of said method said alternate cancer treatment comprising or consisting of: anti-EGFR antibodies, small molecule inhibitors, radiation therapy, and chemotherapy.

The invention is now further explained by means of embodiments resulting in additional features, characteristics and advantages of the invention. The embodiments are of pure illustrative nature and do not limit the scope or range of the invention. The features mentioned in the specific embodiments are general features of the invention which are not only applicable in the specific embodiment but also in an isolated manner and in the context of any embodiment of the invention.

The invention is now described and explained in further detail by referring to the following non-limiting examples and figures.

### BRIEF DESCRIPTION OF THE DRAWINGS

FIG. 1: Consort diagrams of the discovery (A) and confirmation (B) cohorts.

FIG. 2: Workflow for digital image analysis using the QuPath software platform for digital image analysis. (A) Scanning of the slides with tissue sections stained for EGFR using enzymatic immunohistochemistry. (B) Whole slide scans (WSS) are uploaded on the QuPath software platform in a project file. (C) In a first step, WSS are designated as «Brightfield (H-DAB)» type and stain vectors are estimated using the Visual Stain Editor. (D) The region of interest (ROI)—defined as the whole tumor tissue within the stained tissue section—is marked for downstream analysis using the Wand Tool (yellow contour). (E) The Positive Cell Detection tool is used to assess the number and percentage of EGFR-positive cells using hematoxylin as counterstaining and a single intensity threshold for EGFR. (F) Overlay of cell detection mask (positive cells-red mask; negative cells-blue mask). (G) The Train Object Classifier allows for cell classification into EGFR-positive tumor cells (red mask), EGFR-negative tumor cells (blue mask), and non-tumor cells (yellow mask).

FIG. 3: EGFR expression assessed by immunohistochemistry (IHC) and digital image analysis in therapy-naïve metastatic tissue in the discovery cohort. Representative micrographs depicting low EGFR positivity in a therapy-naïve melanoma metastasis from a patient without relapse (upper row) and high EGFR positivity in a therapy-naïve melanoma metastasis from a patient who relapsed (lower row) (both patients from the discovery cohort); left column—whole slide scans of the EGFR-stained metastases (scale bars=2 mm); middle column—40× magnification of the region marked with * in the left column (scale bars=20 μm); right column—an overlay of positive cell detection mask using the QuPath software platform for whole slide digital image analysis (scale bars=20 μm); insets 90× magnification (scale bars=5 μm).

FIG. 4: EGFR expression assessed by immunohistochemistry (IHC) and digital image analysis in therapy-naïve metastatic tissue from patients in the confirmation cohort. Representative micrographs depicting low EGFR positivity in a therapy-naïve melanoma metastasis from a patient without relapse (upper row) and high EGFR positivity in a therapy-naïve melanoma metastasis from a patient who relapsed (lower row); left column-whole slide scans of the EGFR-stained metastases (scale bars=2 mm); middle column-40× magnification of the region marked with * in the left column (scale bars=20 μm); right column—an overlay of positive cell detection mask using the QuPath software platform for whole slide digital image analysis (scale bars=20 μm); insets 90× magnification (scale bars=10 μm).

FIG. 5: EGFR expression in therapy-naïve metastases is associated with relapse. (A) Percentage of EGFR-positive tumor cells in therapy naïve metastases from patients without relapse vs. patients with relapse in the discovery cohort assessed by digital morphometry. (B) Percentage of EGFR-positive tumor cells in therapy naïve metastases from patients without relapse vs. patients with relapse in the confirmation cohort assessed by digital morphometry. Statistical analysis was performed using the Mann-Whitney test.

FIG. 6: EGFR expression in therapy-naïve metastases inversely correlates with relapse-free survival. (A) Kaplan-Meier Curves of relapse-free survival in patients with low EGFR expression vs. high EGFR expression in the discovery cohort and (B) in the confirmation cohort. Statistical significance was assessed using the Log-rank test; EGFR cut-off at 18.37% and 0.048%, respectively, for discovery and confirmation cohorts calculated using ROC curve analysis and Youden's index for EGFR positivity values; (***p=0.0004; *p=0.0225).

FIG. 7: EGFR expression assessed by immunohistochemistry (IHC) and digital image analysis in primary tumors from patients included in the confirmation cohort. Representative micrographs depicting low EGFR positivity in primary melanoma from a patient without relapse (upper row) and high EGFR positivity in a patient with relapse (lower row); left column-whole slide scans of the EGFR-stained metastases (scale bars=1 mm); middle column-40× magnification of the region marked with * in the left column (scale bars=20 μm); right column—an overlay of positive cell detection mask using the QuPath software platform for whole slide digital image analysis (scale bars=20 μm); insets 90× magnification (scale bars=10 μm).

FIG. 8: EGFR expression in primary tumors does not correlates with overall survival. (A) Percentage of EGFR-positive tumor cells in primary tumors from patients without relapse vs. patients with relapse assessed by digital morphometry. (B) Kaplan-Meier Curves of overall survival in patients with low EGFR-expressing vs. high EGFR-expressing primary tumors. Statistical significance was assessed using the Log-rank test; p=0.7286; EGFR cut-off at 0.048% calculated using Yoden's index and ROC curve analysis for EGFR positivity values for the confirmation cohort.

### EXAMPLES

### 1. Material and Methods

### EGFR Enzymatic Immunohistochemistry

Available therapy naïve tissue samples taken for diagnostic histological examination were formalin-fixed and paraffin-embedded (FFPE) using the standard processing protocols. Three-micron thick serial sections were cut using a rotary microtome. For the discovery cohort single epitope enzymatic immunohistochemistry on FFPE tissue for EGFR was performed in the Institute for Pathology of Kantonsspital St. Gallen using a Leica BOND MAX automated immunostainer (Leica Microsystems, CH), and a monoclonal mouse anti-human EGFR (Leica Biosystems, CH, catalog number NCL-EGFR-384, dilution 1:60, HIER-pH 9/30 min/95° C., incubation for 60 min). For the confirmation cohort, single epitope enzymatic immunohistochemistry on FFPE tissue for EGFR was performed in the Institute for Pathology of University Hospital Tuebingen using a Ventana Benchmark XT automated immunostainer (Ventana, DE), and a monoclonal mouse anti-human EGFR (Dako/Agilent, DE, catalog number M7239, dilution 1:40, antigen retrieval—protease ⅛ min, incubation for 32 min at 37° C.).

### Image Analysis

Quantitative morphometry was performed on all stained slides using the QuPath v0.2.3 software platform for whole slide image analysis (Edinburgh, UK) 5. Whole slide scans were individually loaded into the software. For each slide image stain vector (i.e., color) and background estimates were applied to improve stain separation within QuPath by selecting a representative area containing background along with examples of strong nuclear counterstaining and chromogen staining, and applying QuPath's “Estimate stain vectors” command to identify stain vectors within this region. The “Positive Cell Detection” command was then used to identify cells across the selected region of interest (ROI)—defined as the whole tumor tissue within the stained tissue section—based upon nuclear staining. The full extent of each cell was estimated based upon a constrained expansion of the nucleus region, and calculates up to 33 measurements of intensity and morphology, including nucleus area, circularity, staining intensity for counterstaining and chromogen, and nucleus/cell area ratio. A random trees classifier (RTrees) was then interactively trained to distinguish tumor cells from all other detections (comprising non-tumor epithelial cells, immune cells, stromal cells, necrosis, or any artefacts misidentified as cells). Cells were classified as positive or negative based upon a single intensity threshold applied to the maximum optical density of the detection chromogen within the nucleus or cytoplasm of the cell depending on the expression pattern. Summary scores were generated as the percentage of cells classified as positive, with “other” detections removed.

### NGS Analysis

DNA was extracted from tumor and normal samples, and target regions were enriched using a custom sequencing panel with more than 700 cancer-related genes and sequenced on a NovaSeq6000 (Illumina, San Diego, USA). Data were analyzed with the megSAP pipeline, using BWA mem2 for mapping, strelka2 for somatic and freebayes for germline variant calling, ClinCNV for somatic and germline copy-number variants, manta for structural variants, and various databases for variant annotation. Somatic variants were classified according to the VICC criteria and germline variants according to the ACMG guidelines.

### 2. Results

Adjuvant therapy with immune checkpoint inhibitors (ICI) became standard for resected stage III/IV melanoma after the results from the Checkmate 238 and Keynote 054 studies. However, 30-40% of patients relapse despite adjuvant therapy.

Epidermal growth factor receptor (EGFR) expression is linked to melanoma dedifferentiation, acquired resistance to MEK inhibitors, and is targeted in various tumor entities. The inventors reported that EGFR amplification did not correlate with the corresponding levels of EGFR protein in patients progressing under (not responding to) ICI.

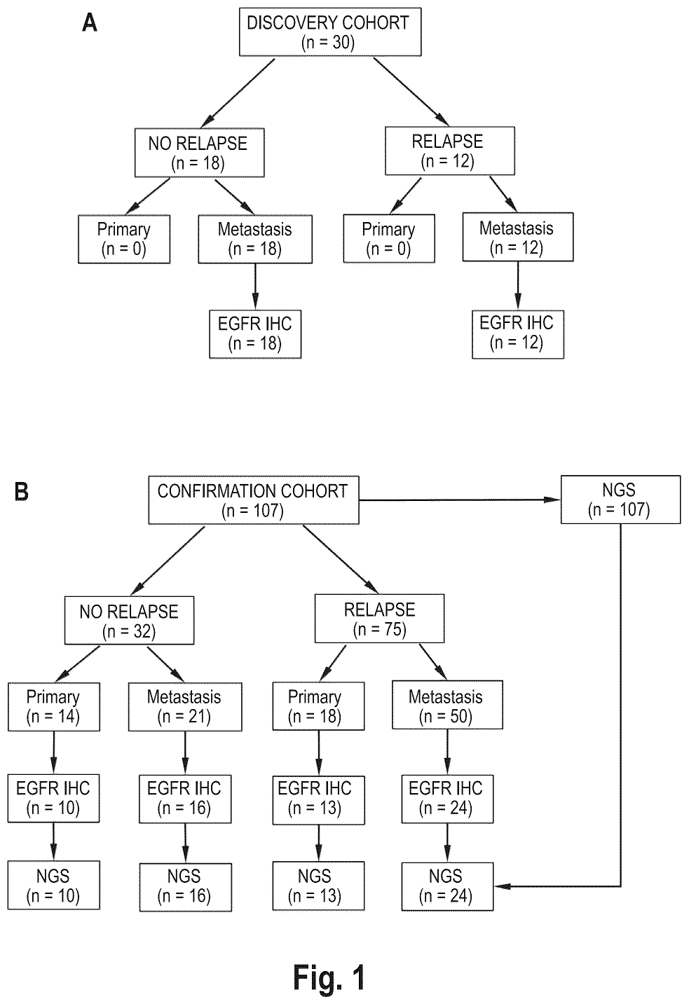

The inventors investigated if EGFR expression could predict relapse to adjuvant ICI in melanoma patients. Two cohorts were included: a discovery cohort from University Hospital Zurich and a confirmation cohort from University Hospital Tuebingen. The inventors analyzed all available samples used for diagnosis (FIG. 1).

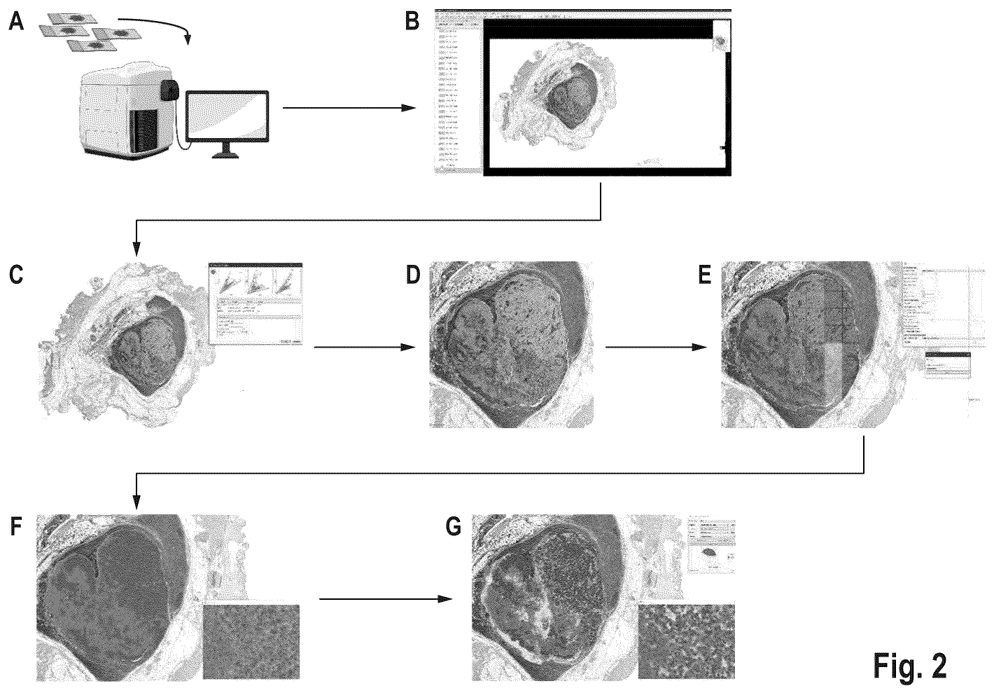

Immunohistochemistry (IHC) for EGFR was performed on formalin-fixed paraffin-embedded tissues. Quantitative digital morphometry was performed using the QuPath v0.2.3 software for image analysis (Bankhead et al. QuPath: Open source software for digital pathology image analysis. Scientific reports. 2017; 7 (1): 16878) (FIG. 2).

A cut-off for EGFR expression that would best identify two groups of patients with higher relapse-free survival (RFS) difference was calculated using ROC curve analysis and Youden's index for EGFR positivity values. RFS and follow-up time (FU) were calculated considering the date of ICI therapy start and relapse date or date of the last contact, respectively. Results are reported as two-sided p values with 95% confidence intervals (95% CI). Statistical significance was set at p<0.05.

The inventors included 137 therapy-naïve patients receiving adjuvant ICI (pembrolizumab or nivolumab). The median FU was 45 and 39 months in the discovery and confirmation cohorts, respectively (95% CI 41.8-48.2 and 36.8-41.2, respectively) (Table 1):

TABLE 1
Clinical characteristics of the patients from
the discovery and confirmation cohorts.

| | Discovery | Confirmation | Chi- |
|---|---|---|---|
| | cohort | cohort | square |
| | (N = 30) (N/%) | (N = 107) (N/%) | test |
| Characteristic | | | |

Age at start of treatment - yrs.

| | | |
|---|---|---|
| IQR | 51-69 | 53-75 |

Sex

| | | | |
|---|---|---|---|
| Female | 14 (47) | 49 (46) | 0.932 |
| Male | 16 (53) | 58 (54) | |

BRAF status

| | | | |
|---|---|---|---|
| WT | 17 (56) | 68 (63) | 0.492 |
| Mutant | 13 (44) | 39 (37) | |

NRAS status

| | | | |
|---|---|---|---|
| WT | 25 (83) | 72 (67) | 0.088 |
| Mutant | 5 (17) | 35 (33) | |

Relapse

| | | | |
|---|---|---|---|
| Yes | 12 (40) | 76 (71) | 0.002 |
| No | 18 (60) | 31 (29) | |

Relapse-free survival (months)*

| | | |
|---|---|---|
| Median (95% CI) | Not reached | 11 (6.8-15.2) |
| 1 y, 2 y and 3-y | 63 (46-80)^{$} | 49 (39-58); 36 |
| RFS rate (95% CI) | | (27-45); 29 (20-38) |

Overall survival (months)^{†}

| | | |
|---|---|---|
| Median | Not reached | Not reached |
| 1 y, 2 y and 3-y | 90 (76-100); | 92 (86-96); 83 |
| OS rate (95% CI) | 87 (74-99)^{&} | (76-90); 76 (67-84) |

*Relapse-free survival was calculated from the date of the first dose of ICI to the date of relapse or censoring of data;
^{$}1 y, 2 y, and 3 y RFS have the same value;
^{†}Overall survival was calculated from the date of the first dose of ICI to the date of death or censoring of data;
^{&}2 y and 3 y OS rate are the same.

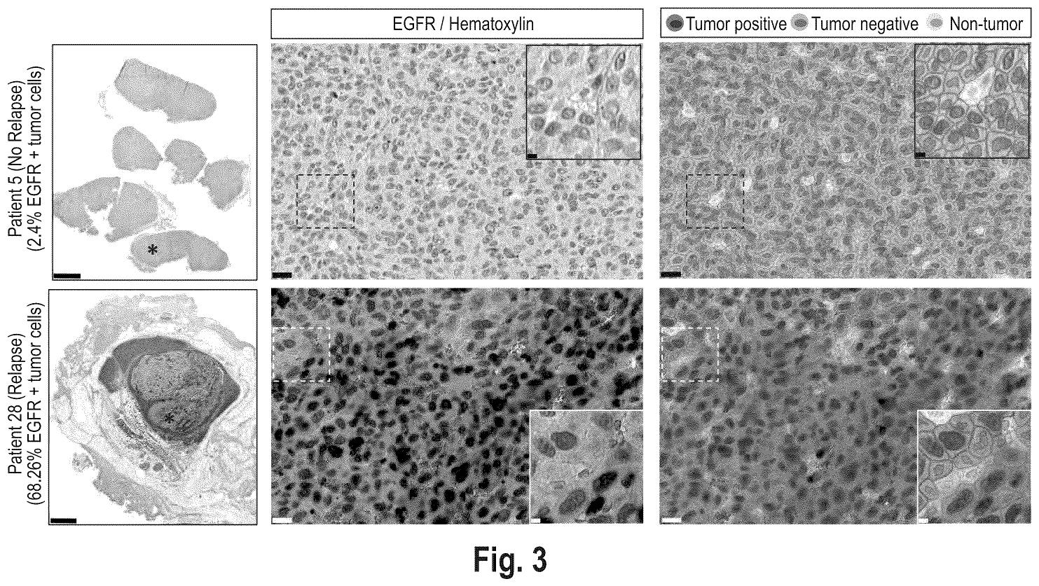

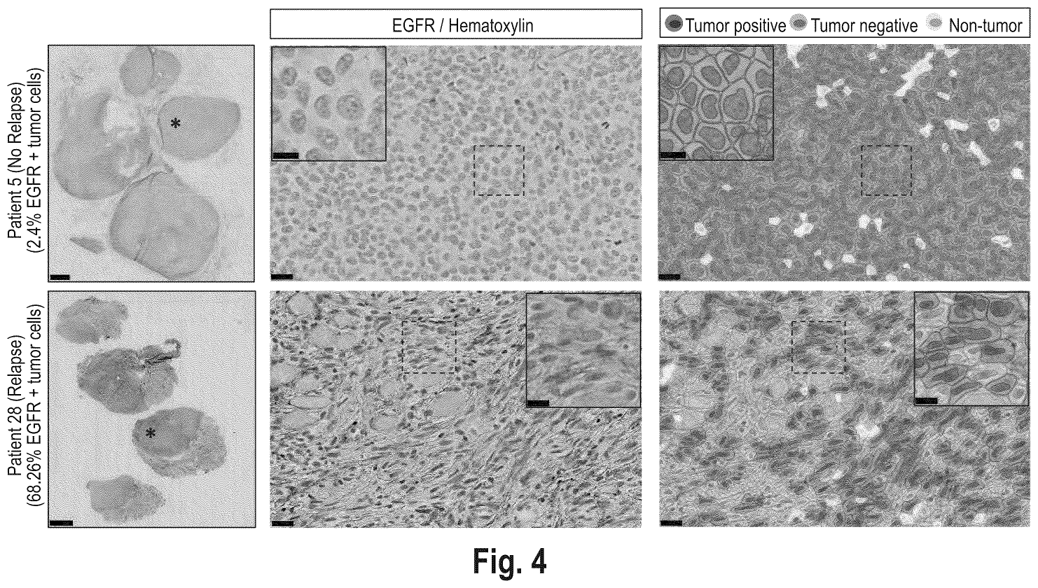

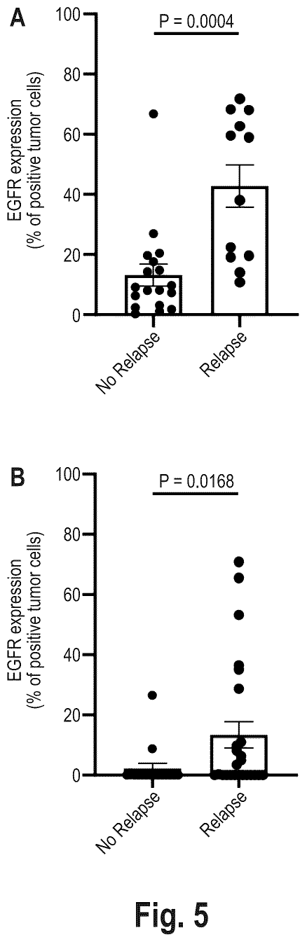

A total of 71 (52%) therapy naïve lymph nodes, in-transit or cutaneous metastases, and 23 (17%) primary tumors were available for evaluation of EGFR expression (FIG. 1). In both cohorts, patients with high EGFR expression in therapy-naïve metastatic tissue had a significantly higher relapse rate than patients with low EGFR expression (p-0.0004 and p-0.0168 for the discovery and confirmation cohorts, respectively) (FIG. 3, FIG. 4, FIG. 5).

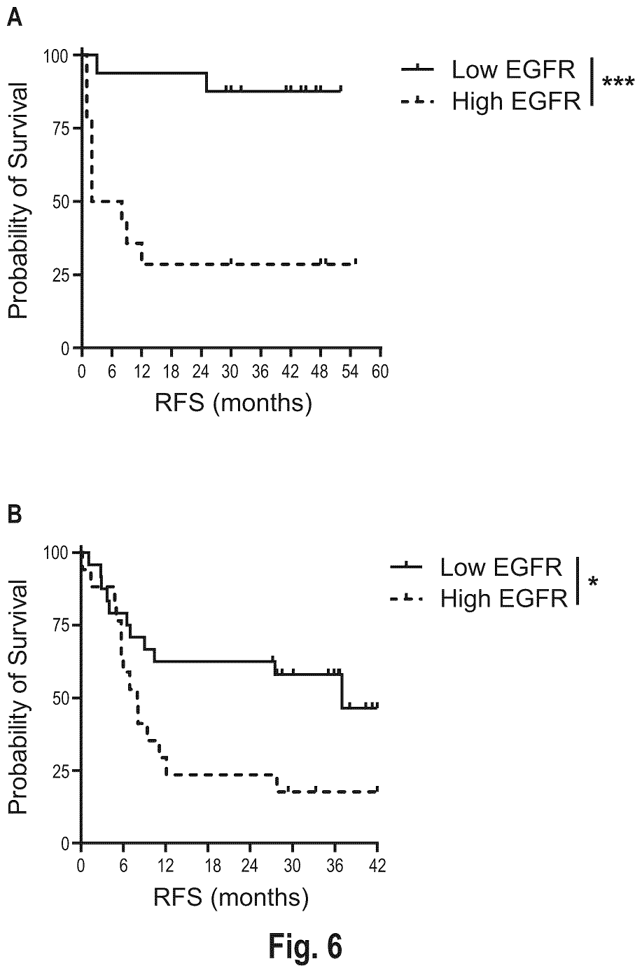

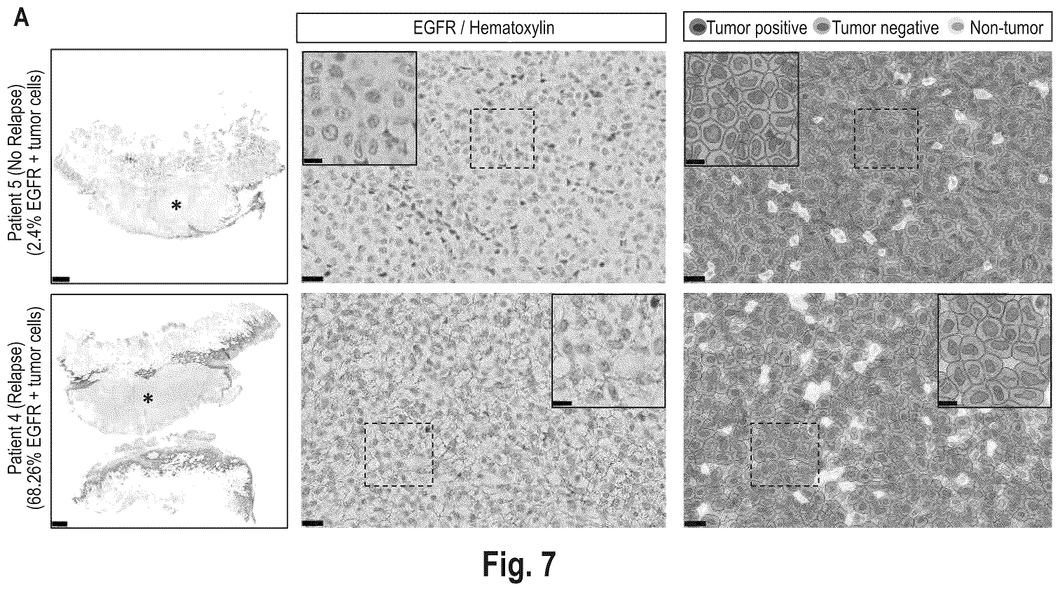

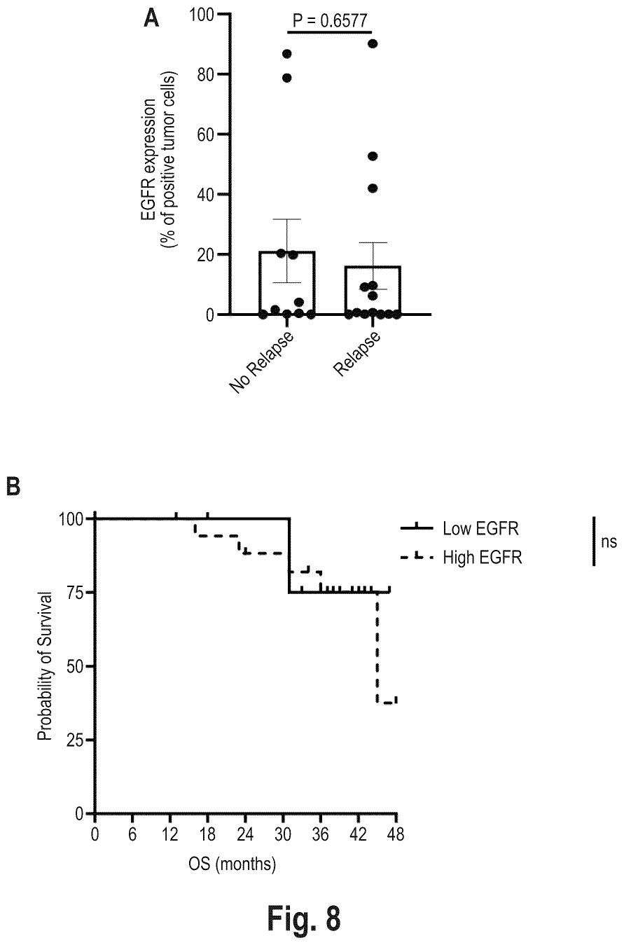

The optimal cut-offs for EGFR expression in therapy-naïve metastatic tissue were 18.37% and 0.048% for discovery and confirmation cohorts, respectively (FIG. 6 A-B). In both cohorts, patients with high EGFR expression had significantly worse outcomes. In the discovery cohort, the median RFS for these patients was three months; in patients with low EGFR expression, the median RFS was not reached (95% CI: 0.00-11.6 and not reached; p=0.0004). Similar results were seen in the confirmation cohort; the median RFS was eight months in the subgroup with high EGFR expression and not reached in the subgroup with low EGFR expression (95% CI: 5.3-10.6 and not reached; p=0.0225) (FIG. 6). EGFR expression in primary tumors did not correlate with survival (FIG. 7, FIG. 8). NGS analysis of 40 therapy naïve metastases showed that EGFR amplification did not correlate with the corresponding levels of EGFR protein (Table 2).

TABLE 2
EGFR expression assessed by IHC in relation to EGFR
mutations or amplifications identified using NGS analysis
in therapy naïve metastatic tissue.

| | EGFR IHC | EGFR amplifications |
|---|---|---|
| Study ID | (% positive tumor cells) | (≥3 copies) and/or mutations |
| 1 | 0.44 | — |
| 2 | 0 | — |
| 4 | 8.22 | amplification |
| 7 | 6.50 | amplification |
| 8 | 0 | amplification |
| 16 | 0.32 | — |
| 17 | 10.95 | — |
| 19 | 4.96 | amplification |
| 23 | 0 | — |
| 24 | 0 | amplification |
| 28 | 0 | amplification |
| 30 | 9.78 | amplification |
| 32 | 70.91 | amplification |
| 34 | 65.58 | — |
| 35 | 3.46 | amplification |
| 36 | 0 | — |
| 37 | 0 | — |
| 42 | 26.58 | — |
| 45 | 0 | — |
| 47 | 0 | amplification |
| 48 | 0.10 | amplification |
| 58 | 0 | — |
| 59 | 0 | amplification |
| 60 | 28.68 | amplification |
| 69 | 0 | amplification |
| 70 | 0 | amplification |
| 72 | 0 | — |
| 73 | 0 | — |
| 74 | 35.14 | amplification |
| 75 | 53.17 | — |
| 79 | 0 | amplification |
| 81 | 0 | amplification |
| 84 | 0 | — |
| 85 | 0 | amplification |
| 86 | 36.57 | amplification |
| 87 | 0 | amplification |
| 88 | 8.76 | — |
| 90 | 0 | — |
| 92 | 0 | amplification |
| 102 | 0 | — |

Using two independent cohorts, the inventors showed that EGFR expression is positively associated with relapse in patients with melanoma treated with adjuvant ICI.

Here the inventors identified EGFR expression in therapy-naïve metastatic tissue as a potential negative predictive factor for adjuvant ICI.

1. A method of predicting a patient's benefit from therapy with an immune checkpoint inhibitor (ICI), comprising the steps of:
1) Providing a biological sample from the patient;
2) Determining the expression level of epidermal growth factor receptor (EGFR) on the biological material to obtain C_{EGFR}^{Pat.};
3) Comparing C_{EGFR}^{Pat.} with the expression level of EGFR in a reference biological sample C_{EGFR}^{Ref.};
4) Predicting
no therapeutic benefit if C_{EGFR}^{Pat.}>C_{EGFR}^{Ref.},
a therapeutic benefit if C_{EGFR}^{Pat.}≤C_{EGFR}^{Ref.}.

2. (canceled)

3. The method of claim 1, wherein the biological sample comprises primary melanoma tissue or metastatic melanoma tissue.

4. (canceled)

5. The method of claim 1, wherein in step (4) no therapeutic benefit is predicted if at least 5% of the cells of the biological sample from the patient express EGFR.

6. The method of claim 1, wherein the ICI is an anti-PD-1 antibody or an anti-CTLA4 antibody.

7.-9. (canceled)

10. The method of claim 1, wherein said immune checkpoint inhibitor is selected from the group consisting of: pembrolizumab, nivolumab, ipilimumab, tremelimumab, cemiplimab, spartalizumab, atezolizumab, durvalumab, and avelumab, pembrolizumab, and nivolumab.

11. (canceled)

12. The method of claim 1, wherein the patient is suffering from melanoma.

13. (canceled)

14. A method of treatment of a cancer patient with an immune checkpoint inhibitor (ICI) including the method of claim 1, the method comprising the steps of:
1) Providing a biological sample from the patient;
2) Determining the expression level of epidermal growth factor receptor (EGFR) on the biological material to obtain C_{EGFR}^{Pat.};
3) Comparing C_{EGFR}^{Pat.} with the expression level of EGFR in a reference biological sample C_{EGFR}^{Ref.}
4) Refraining from administering the ICI to the patient if C_{EGFR}^{Pat.}>C_{EGFR}^{Ref.};
or
Administering the ICI to the patient if C_{EGFR}^{Pat.}≤C_{EGFR}^{Ref.}.

15. The method of claim 14, wherein the biological sample comprises skin cancer tissue.

16. The method of claim 14, wherein the biological sample comprises primary melanoma tissue or metastatic melanoma tissue.

17. (canceled)

18. The method of claim 14, wherein in step (4) it is refrained from administering the ICI if at least 5% of the cells of the biological sample from the patient express EGFR.

19. The method of claim 14, wherein the ICI is an anti-PD-1 antibody.

20. The method of claim 14, wherein the ICI is pembrolizumab or nivolumab.

21. (canceled)

22. The method of claim 14, wherein the ICI is an anti-CTLA4 antibody.

23. The method of claim 14, wherein said immune checkpoint inhibitor is selected from the group consisting of: pembrolizumab, nivolumab, ipilimumab, tremelimumab, cemiplimab, spartalizumab, atezolizumab, durvalumab, and avelumab.

24. The method of claim 14, wherein the expression level is determined by immunohistochemistry (IHC).

25. The method of claim 14, wherein the patient is suffering from melanoma.

26. The method of claim 14, wherein the patient is suffering from stage III/IV melanoma.

27. A method of treatment of a patient with metastatic melanoma, comprising the steps of:
measuring the expression level of epidermal growth factor receptor (EGFR) in metastatic melanoma cells from the patient to obtain C_{EGFR}^{Pat.};
comparing C_{EGFR}^{Pat.} with the expression level of EGFR in a reference biological sample C_{EGFR}^{Ref.}
predicting that the patient will benefit from treatment with an immune checkpoint inhibitor (ICI) if C_{EGFR}^{Pat.} Is less than or equal to C_{EGFR}^{Ref.};
subjecting the patient to a cancer treatment regimen that comprises administering the ICI to the patient.

28. A method of treatment of patients with metastatic melanoma, the method comprising:
A. for each patient,
i. measuring the expression level of epidermal growth factor receptor (EGFR) in metastatic melanoma cells from the patient to obtain C_{EGFR}^{Pat.}; and
ii. comparing C_{EGFR}^{Pat} with the expression level of EGFR in a reference biological sample C_{EGFR}^{Ref};
B. treating the patients for melanoma, wherein
i. for at least one of the patients, predicting that the at least one patient will benefit from treatment with an immune checkpoint inhibitor (ICI) if C_{EGFR}^{Pat} of said at least one patient is less than C_{EGFR}^{Ref.}; and treating said at least one patient with a cancer treatment regimen that comprises administering the ICI to the patient; and
ii. for at least one other of the patients, predicting that the at least one other of the patients will not benefit from treatment with an immune checkpoint inhibitor (ICI) if C_{EGFR}^{Pat.} of said at least one other patient is equal or greater than C_{EGFR}^{Ref}; and treating said at least one other patient with an alternate cancer treatment regimen that does not include the ICI.

29. The method of claim 28, wherein said alternate cancer treatment comprising or consisting of: anti-EGFR antibodies, small molecule inhibitors, radiation therapy, and chemotherapy.

30. (canceled)
